(19) United States
(12) Patent Application Publication
(10) Pub. No.: US 2020/0005944 A1
(43) Pub. Date: Jan. 2, 2020

(54) DEVICE AND METHOD FOR HEALTH INFORMATION PREDICTION USING BIG DATA

(71) Applicant: DNESOFTDIGM CO., LTD, Pohang-si (KR)

(72) Inventor: Dae Ho LEE, Pohang-si (KR)

(21) Appl. No.: 16/465,543

(22) PCT Filed: Dec. 5, 2017
(86) PCT No.: PCT/KR2017/014159

§ 371 Date: Jun. 24, 2019

(30) Foreign Application Priority Data
Dec. 6, 2016 (KR) 10-2016-0164949
Feb. 7, 2017 (JP) 10-2017-0016988

(51) Int. Cl. G16H 50/30; G16H 10/60; G16H 70/60

(52) U.S. Cl. CPC G16H 50/30; G16H 70/60; G16H 10/60

(57) ABSTRACT

Disclosed is a health information prediction device which calculates a disease-specific health score through an open-type personal health record platform corresponding to big data, performs comparison and analysis of environmental groups similar to a user, and provides exercise and stress scores, medical service assessment information of a medical institution, and an individual health indicator. By the health information prediction device of the present invention, a user can expect self-health care and life improvement.

### TECHNICAL FIELD

The present invention relates to a device and method for health information prediction using big data, and more specifically, to a predicting device and method for providing self-health care using big data.

### BACKGROUND ART

Big data is a technology for effectively and rapidly collecting and analyzing huge amounts of data that exceed the level of conventional relationship database processing. Major countries and global companies are focusing on fostering and utilizing big data industries, and about 30% of companies are using big data for business directly or indirectly, and the proportion of big data used in companies is expected to gradually rise.

The National Institutes of Health in the U.S. is attempting a medical reform through the Pillbox project using big data such as a medicine search site operated by the National Library of Medicine. The site allows statistical data of various diseases of the users, which are obtained through medicine search services, to be utilized so that major diseases to be controlled are managed and predicted and also allows the distribution and trend of diseases to be predicted so that an early response at a national level is performed.

In South Korea, problems of aging and chronic diseases are also rapidly increasing. There is a trend of a transition from post-treatment to providing preventive medical services. Accordingly, there is a growing interest in management and prediction of health and diseases, which is leading to a gradually increasing demand for a service, such as the medicine search sites of The National Institutes of Health.

### DISCLOSURE

### Technical Problem

The present invention is directed to providing a device for predicting heath information through big data.

The present invention is directed to providing a method of predicting heath information through big data.

### Technical Solution

One aspect of the present invention provides a device for predicting health information including a health examination unit configured to derive health examination data using an open-type personal health record (PHR) platform corresponding to big data, a diagnosis unit configured to derive diagnostic data using the open-type PHR platform, and a health score calculation unit configured to calculate a disease-specific health score and a personal health score.

The device for predicting health information may further include a future health score calculation unit configured to calculate a disease-specific future health score of a user.

The device for predicting health information may further include a health management unit configured to derive an individual health indicator on the basis of the personal health score and the disease-specific future health score.

The device for predicting health information may further include a comparison analysis unit configured to perform comparison and analysis on an environment group similar to a user on the basis of the disease-specific health score.

The device for predicting health information may further include a service evaluation unit configured to derive medical service evaluation information of a medical institution.

The device for predicting health information according to the present invention may further include a personal health record (PHR) platform.

The PHR platform may include an information collecting unit configured to collect health and disease management knowledge, a feedback unit configured to perform feedback with reference to a knowledge storage, a personal life health information record interface, an application programming interface (API) for service system integration and linkage, a data linking unit configured to link typical data and atypical data, a cloud service unit configured to provide a cloud computing service using big data, and a health predicting unit configured to predict individual health care through converting a personal health record into public data.

The device for predicting health information according to the present invention may further include: a u-health care unit configured to provide real time u-health care using health information related data; a protected health information (PHI) monitor unit configured to provide monitoring of PHI using health insurance big data; a disease prevention unit configured to provide a disease prevention program through analysis of disease data; a medical service unit configured to provide a health and medical service including a diagnosis, a treatment, and follow-up care using the u-health care; a data management unit configured to perform integrated data management for providing a recommended service through a vaccination time and existing personal health record material that is linked with vaccination data; a prediction model unit configured to provide a health-prediction-likelihood model through analysis of a health examination result, a treatment, and an administration history; and a disease analysis unit configured to analyze a probability of a disease outbreak through integration of personal genetic information and health type information.

The device for predicting health information according to the present invention may further include an exercise management unit configured to collect physical information and exercise information of a user, calculate an exercise score on the basis of an individual exercise practice indicator formula, and assess a degree of exercise according to the calculated exercise score.

The device for predicting health information according to the present invention may further include an absolute stress management unit configured to collect stress information of a user, calculate an absolute stress score according to an individual absolute stress indicator formula, and assess a degree of stress of the user according to the calculated absolute stress score.

The device for predicting health information according to the present invention may further include a relative stress management unit configured to calculate a relative stress score according to an individual relative stress indicator formula and assess a degree of stress of the user according to the calculated relative stress score.

Another aspect of the present invention provides a method of predicting health information, the method including deriving health examination data and diagnostic data using an open-type PHR platform corresponding to big data, and a disease-specific score determinant from the open-type PHR platform and calculating a disease-specific health score.

The method of predicting health information may further include calculating a personal health score on the basis of the health examination data and the diagnostic data.

The method of predicting health information may further include calculating a disease-specific future health score.

The method of predicting health information may further include deriving an individual health indicator on the basis of the personal health score and the disease-specific future health score.

The method of predicting health information may further include performing comparison and analysis on an environment group similar to a user on the basis of the disease-specific health score.

The method of predicting health information may further include deriving medical service evaluation data of a medical institution.

The method of predicting health information according to the present invention may further include constructing a personal health record (PHR) platform.

The constructing of the PHR platform may include collecting health and disease knowledge, performing feedback with reference to a knowledge storage, using a personal life health information record interface, using an application programming interface (API) for service system integration and linkage, linking typical data and atypical data, providing a cloud computing service using big data, and predicting individual health care through converting a personal health record into public data.

The method of predicting health information according to the present invention may further include: providing real time u-health care using health information related data; monitoring protected health information (PHI) using health insurance big data; providing a disease prevention program through analysis of disease data; providing a health and medical service including a diagnosis, a treatment, and follow-up care using the u-health care; providing integrated data management for providing a recommended service through a vaccination time and existing personal health record material that is linked with vaccination data; providing a health-prediction-likelihood model through analysis of a health examination result, a treatment, and an administration history; and analyzing a probability of a disease outbreak through integration of personal genetic information and health type information.

The method of predicting health information may further include collecting physical information and exercise information of a user, calculating an exercise score according to an individual exercise practice indicator formula, and assessing a degree of exercise of the user according to the exercise score.

The method of predicting health information may further include collecting stress information of a user, calculating an absolute stress score according to an individual absolute stress indicator formula, and assessing a degree of stress of the user according to the calculated absolute stress score.

The method of predicting health information may further include collecting the absolute stress score, calculating a relative stress score according to an individual relative stress indicator formula, and assessing a degree of stress of the user according to the calculated relative stress score.

### Advantageous Effects

According to the present invention, personal health information can be managed to thereby provide an individual health indicator that is utilized for self-health care.

According to the present invention, service evaluation information of medical institutions can be provided and utilized when selecting a medical institution.

In addition, according to the present invention, exercise and stress of busy workers can be managed so that living conditions of workers are expected to be improved.

### DESCRIPTION OF DRAWINGS

FIG. 1 is a graph showing a result of a Google Flu Trend (GFT) service through a big data analysis technique.

FIG. 2 is a schematic block diagram illustrating a device for health information prediction according to an embodiment of the present invention.

FIG. 3 is a block diagram illustrating a device for health information prediction according to an embodiment of the present invention.

FIG. 4 is a flowchart showing a method of calculating a disease-specific health score according to the present invention.

FIG. 5 is an output screen of an individual health indicator derived by a health care unit according to an embodiment of the present invention.

FIG. 6 is a flowchart showing a method of assessing the degree of exercise according to the present invention.

FIG. 7 is an operational flowchart showing a method of assessing the degree of absolute stress according to the present invention.

FIG. 8 is an operational flowchart showing a method of assessing the degree of relative stress according to the present invention.

### BEST MODES OF THE INVENTION

One aspect of the present invention provides a device for health information prediction, the device including a health examination unit configured to derive health examination data using an open-type personal health record (PHR) platform corresponding to big data, a diagnosis unit configured to derive diagnosis data using the open-type PHR platform, and a health score calculation unit configured to calculate a disease-specific health score and a personal health score.

Another aspect of the present invention provides a method for health information prediction, the method including deriving health examination data and diagnostic data from an open-type personal health record (PHR) platform corresponding to big data and calculating a health score for each disease.

### Modes of the Invention

Example embodiments of the present invention are disclosed herein. However, specific structural and functional details disclosed herein are merely representative for purposes of describing example embodiments of the present invention, and example embodiments of the present invention may be embodied in many alternative forms and should not be construed as limited to example embodiments of the present invention set forth herein.

Accordingly, while the invention is susceptible to various modifications and alternative forms, specific embodiments thereof are shown by way of example in the drawings and will herein be described in detail. It should be understood, however, that there is no intent to limit the invention to the particular forms disclosed, but on the contrary, the invention is to cover all modifications, equivalents, and alternatives falling within the spirit and scope of the invention. Like numbers refer to like elements throughout the description of the figures.

It will be understood that, although the terms first, second, etc. may be used herein to describe various elements, these elements should not be limited by these terms. These terms are only used to distinguish one element from another. For example, a first element could be termed a second element, and, similarly, a second element could be termed a first element, without departing from the scope of the present invention. As used herein, the term “and/or” includes any and all combinations of one or more of the associated listed items.

It will be understood that when an element is referred to as being “connected” or “coupled” to another element, it can be directly connected or coupled to the other element or intervening elements may be present. In contrast, when an element is referred to as being “directly connected” or “directly coupled” to another element, there are no intervening elements present. Other words used to describe the relationship between elements should be interpreted in a like fashion (i.e., “between” versus “directly between”, “adjacent” versus “directly adjacent”, etc.).

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises”, “comprising,”, “includes” and/or “including”, when used herein, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

It should also be noted that in some alternative implementations, the functions/acts noted in the blocks may occur out of the order noted in the flowcharts. For example, two blocks shown in succession may in fact be executed substantially concurrently or the blocks may sometimes be executed in the reverse order, depending upon the functionality/acts involved.

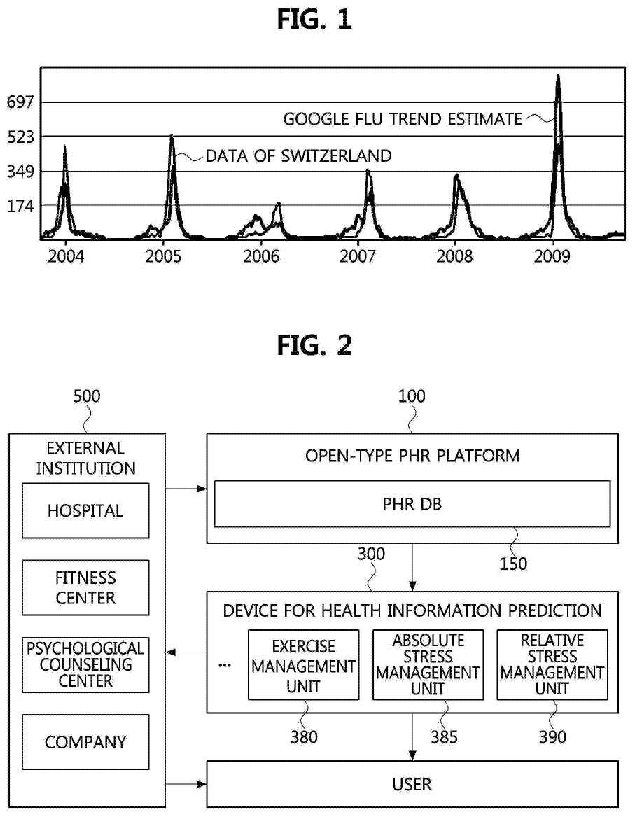

FIG. 1 is a graph showing a result of a Google Flu Trend (GFT) service through a big data analysis technique.

Google, a global company, has started a trend service with the increase in the companies that use big data directly or indirectly for business. The trend service represents a big-data-based service that charts a trend of search keywords in the Google website and shows the chart in real time.

A flu trend service may be an example of the trend service. The flu trend service is a service that informs of a country-specific flu risk level and a state of flu epidemics worldwide and shows a worldwide flu trend through a big data analysis scheme by comparing the number of flu-related search words with the existing flu monitoring system.

FIG. 1 shows graphs showing the level of a flu epidemic in Switzerland predicted by the flu trend service, in which a blue line represents Google's flu trend estimates and a yellow line represents actual data of Switzerland. Referring to FIG. 1, it can be seen that the flu trend estimates are similar to the actual data of Switzerland. This result suggests that prediction through big data has high accuracy.

FIG. 2 is a schematic block diagram illustrating a device for health information prediction according to an embodiment of the present invention.

The block diagram for the operation of the device for health information prediction according to the embodiment of the present invention includes an open-type personal health record (PHR) platform 100, a PHR database 150, a device 300 for health information prediction, and an external institution 500.

The open-type PHR platform 100 may include the PHR database 150 and may be a PHR platform of another company or another institution. In addition, the open-type PHR platform 100 may be serviced by directly constructing a health and disease management knowledge database, in which a closed-type PHR platform may be serviced.

The health and disease management knowledge database may include an information collecting unit configured to collect health and disease management knowledge, a feedback unit configured to perform feedback with reference to a knowledge storage, a personal life health information record interface, an application programming interface (API) for service system integration and linkage, a data linking unit configured to link typical data and atypical data, a cloud service unit configured to provide a cloud computing service using big data, and a health predicting unit configured to predict individual health care through converting a personal health record into public data.

The open-type PHR platform 100 may collect personal health information from the external institution 500 and store the collected personal health information in the PHR database 150. The external institution 500 may include a hospital, a fitness center, a psychological counseling center, and a company, and the personal health information may include examination data, diagnostic data, biosignals, physical information, exercise information, and stress information.

The device 300 for health information prediction may include an exercise management unit 380, an absolute stress management unit 385, and a relative stress management unit 390.

In addition, the device 300 for health information prediction may include a u-health care unit configured to provide real time u-health care using health information related data, a protected health information (PHI) monitor unit configured to provide monitoring of PHI using health insurance big data, a disease prevention unit configured to provide a disease prevention program through a disease data analysis, a medical service unit configured to provide a health and medical service including a diagnosis, a treatment, and follow-up care using the u-health care, a data management unit configured to perform integrated data management for providing a recommended service through a vaccination time and existing PHR material that is linked with vaccination data, a prediction model unit configured to provide a health-prediction-likelihood model through analysis of a health examination result, a treatment, and an administration history, and a disease analysis unit configured to analyze a probability of a disease outbreak through integration of personal genetic information and health type information.

The device 300 for health information prediction may derive the personal health information through a service of the open-type PHR platform 100 including the PHR database 150 or a service of the PHR platform including the directly constructed health and disease management knowledge database.

The device 300 for health information prediction may provide a user with information acquired using the personal health information, that is, a disease-specific health score, a personal health score, a degree of exercise, a degree of stress, and the like, and provide the external institution 500 with the information acquired using the personal health information such that the external institution 500 provides a user with a customized service.

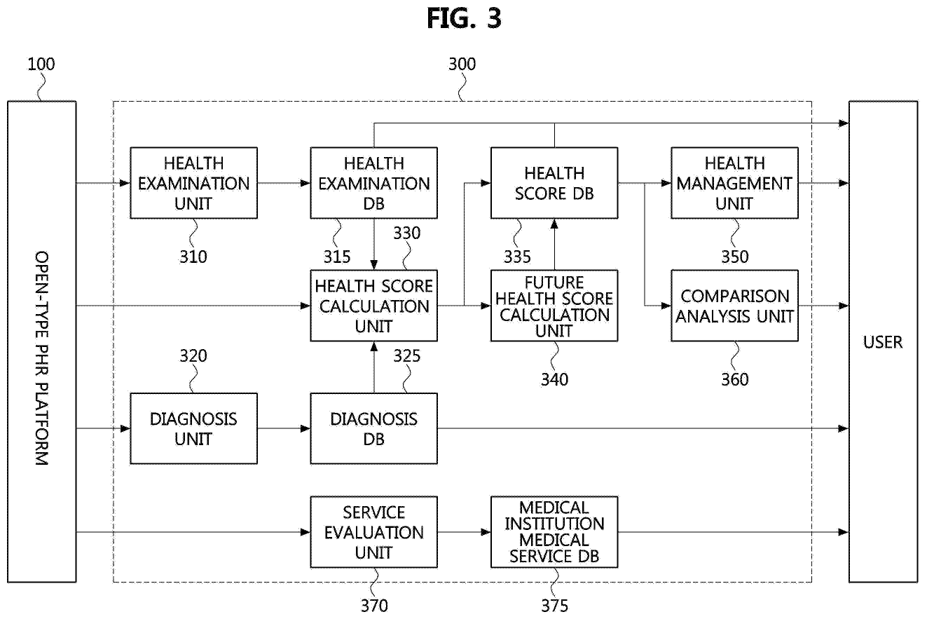

FIG. 3 is a block diagram illustrating the device 300 for health information prediction according to the embodiment of the present invention.

Referring to FIG. 3, the device 300 for health information prediction includes a health examination unit 310, a health examination database (DB) 315, a diagnosis unit 320, a diagnosis DB 325, a health score calculation unit 330, a future health score calculation unit 340, a health score DB 335, a health management unit 350, a comparison analysis unit 360, a service evaluation unit 370, and a medical institution medical service DB 375.

The health examination unit 310 may derive health examination data of the user from the open-type PHR platform 100. The health examination DB 315 may store the health examination data and provide the user with the health examination data.

The diagnosis unit 320 may derive diagnostic data and biosignals of the user from the open-type PHR platform 100. The diagnosis DB 325 may store the diagnostic data and the biosignals and provide the user with the diagnostic data and the biosignals.

The health score calculation unit 330 may extract the health examination data from the health examination DB 315 and extract the diagnostic data from the diagnosis

DB 325. In addition, the health score calculation unit 330 may extract biosignals and information of the external institution from the open-type PHR platform 100 and calculate a disease-specific health score and a personal health score on the basis of the biosignals and the information of the external institution.

The personal health score may be calculated by a personal health score formula including a plurality of disease-specific health scores. The personal health score formula may use a formula of summing disease-specific health scores, a formula of averaging disease-specific health scores, or a formula of summing disease-specific health scores given different weights and dividing the sum by the number of the diseases.

The future health score calculation unit 340 may extract the health examination data, the diagnostic data, the biosignals, and the information of the external institution from the health score calculation unit 330 and calculate a disease-specific future health score on the basis of the health examination data, the diagnostic data, the biosignals, and the information of the external institution.

The health score DB 335 may store the disease-specific health score and the personal health score acquired from the health score calculation unit 330 and provide the user with the disease-specific health score and the personal health score. In addition, the health score DB 335 may store the disease-specific future health score acquired from the future health score calculation unit 340 and provide the user with the disease-specific future health score.

The health management unit 350 may extract the disease-specific health score, the personal health score, and the disease-specific future health score from the health score DB 335, derive an individual health indicator on the basis of the disease-specific health score, the personal health score, and the disease-specific future health score, and provide the user with the individual health indicator.

The comparison analysis unit 360 may extract a personal health score from the health score DB 335 and perform comparison and analysis on an environment group similar to the user on the basis of the personal health score.

The service evaluation unit 370 may derive medical service evaluation information of a medical institution from the open-type PHR platform 100. The medical institution medical service DB 375 may store the medical service evaluation information of the medical institution and provide the user with the medical service evaluation information of the medical institution.

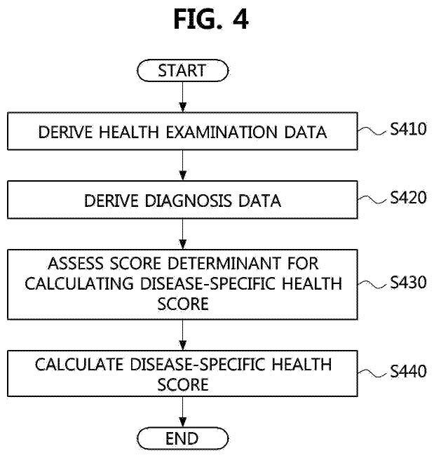

FIG. 4 is a flowchart showing a method of calculating a disease-specific health score according to the present invention.

The method of calculating a disease-specific health score shown in FIG. 4 may be performed by the health score calculation unit 330 shown in FIG. 3, but the agent of the operation is not limited thereto.

Referring to FIG. 4, the method of calculating a disease-specific health score includes deriving health examination data from the open-type PHR platform (S410), deriving diagnostic data from the open-type PHR platform (S420), assessing a score determinant for calculating a disease-specific health score (S430), and calculating a disease-specific personal health score according to the score determinant on the basis of the health examination data and the diagnostic data (S440).

In detail, an example of a method of calculating a health score of hypertension will be described. First, health examination data of the user regarding hypertension is derived from the open-type PHR platform in operation S410, and diagnosis data regarding hypertension is derived from the open-type PHR platform in operation S420. In addition, referring to Table 1, score determinants for calculating a health score of hypertension are assessed with reference to big data of the open-type PHR platform as gender, age, income quintile, family history of hypertension, family history of cancer, body mass index (BMI), daily smoking amount, and one-time alcohol consumption in operation S430.

TABLE 1
HEALTH SCORE DETERMINANTS OF HYPERTENSION

Gender
Age
Income quintile
Family history of hypertension
Family history of cancer
BMI
Daily smoking amount
One-time alcohol consumption
. . .

When the score determinants are assessed, corresponding data is matched according to the score determinants using the health examination data of the user regarding hypertension and the diagnostic data regarding hypertension as shown in Table 2.

TABLE 2
HONG GIL-DONG DATA

| | |
|---|---|
| Gender | male |
| Age | 60 |
| Income quintile | 13 |
| Family history | hypertension |
| BMI | 23 |
| Daily smoking amount | 0 |
| One-time alcohol | 7 glasses of soju |
| consumption | |
| . . . | . . . |

$$score = \frac{100}{1 + exp ( {\sum }_{i} {α}_{i} {X}_{i} )} \qquad [ Equation 1 ]$$

After each piece of data is matched according to the score determinant, the disease-specific health score is calculated through Equation 1. In Equation 1, αᵢ denotes the weight for a score determinant, and Xᵢ denotes a score determinant.

$$\frac{( 100 )}{\begin{matrix}1 + exp ( + 7.76 \times gender ( male \text{:} 1 ; female \text{:} 0 ) + 2.96 \times \\ age ( 60 ) + 1.83 \times hypertension\_familyhistory \\ ( existence \text{:} 1 ; nonexisten \text{: 0} ) … )\end{matrix}} \qquad [ Equation 2 ]$$

When the case of hypertension described above is substituted for Equation 1, a health score for hypertension is calculated as in Equation 2 (S440). The device for health information prediction according to the embodiment of the present invention may be applied to various other diseases, such as diabetes and obesity, in addition to the above described hypertension, to calculate the health score.

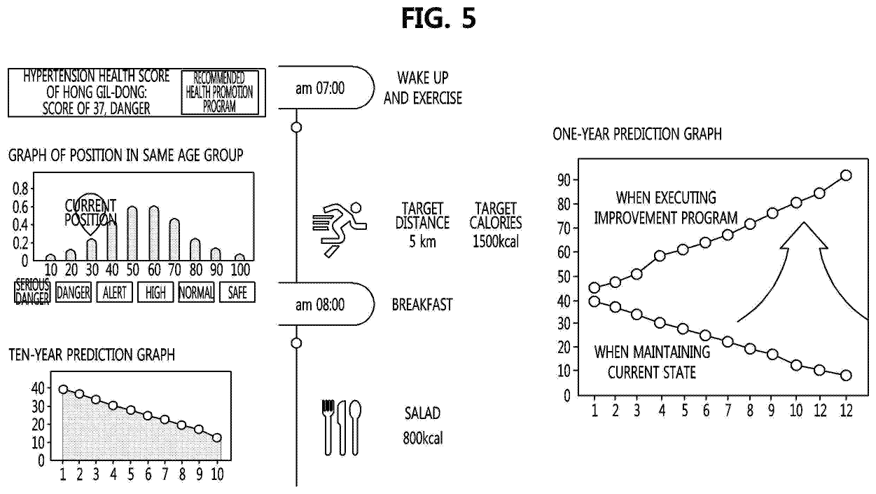

FIG. 5 is an output screen of an individual health indicator derived by the health management unit 350 according to the embodiment of the present invention.

Referring to FIG. 5, a customized health indicator according to the present invention may be provided with a disease-specific health score, a ten-year prediction graph based on a disease-specific future health score, a customized exercise, and a future prediction graph according to the customized exercise.

Items provided by the customized health indicator according to the present invention may include a personal health score, a disease-specific future health score, health examination data, diagnostic data, a comparison analysis result of the same group, and medical service evaluation information of a medical institution.

However, the items provided by the customized health indicator according to the present invention are not limited to the above described items.

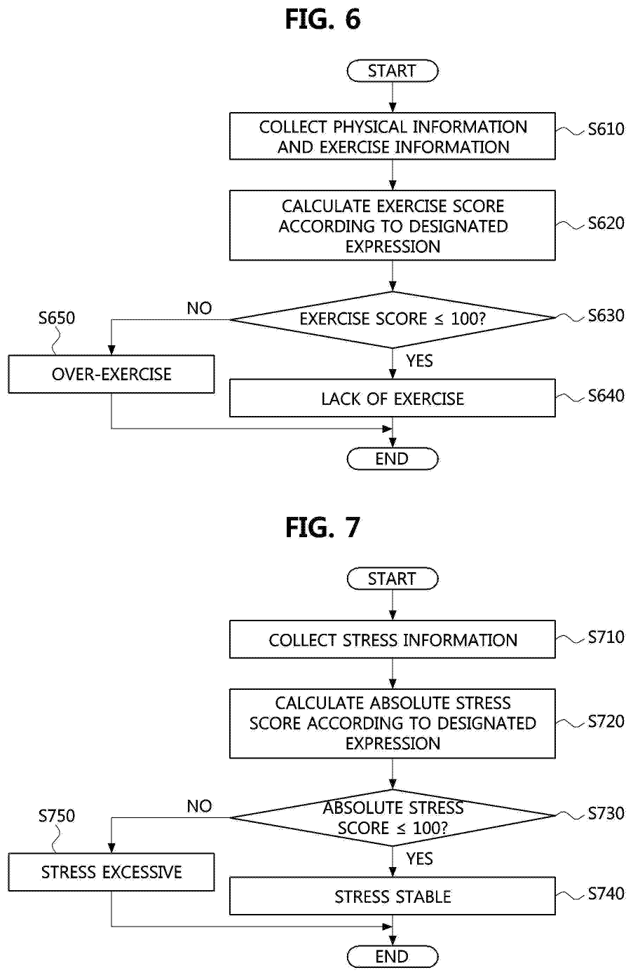

FIG. 6 is a flowchart showing a method of assessing the degree of exercise according to the present invention.

As described above, the device 300 for health information prediction may include an exercise management unit 380 for determining the degree of exercise on the basis of physical information and exercise information of the user. The method of determining the degree of exercise shown in FIG. 6 may be performed by the exercise management unit 380 shown in FIG. 2, but the agent of the operation is not limited thereto.

The method of assessing the degree of exercise using the exercise management unit 380 includes collecting physical information and exercise information of a user from the open-type PHR platform 100 or from the directly servicing PHR platform (S610), calculating an exercise score according to a designated expression shown in Equation 3 (S620), determining whether the exercise score is less than or equal to 100 (S630), estimating lack of exercise when the exercise score is less than or equal to 100 (S640), and estimating over-exercise when the exercise score is greater than 100 (S650).

$$\begin{matrix}exercise\_score = {E}_{p} \times 100 & [ Equation 3 ] \\ {E}_{p} = {β}_{1} \times \frac{{b}_{s}}{{b}_{r}} + {β}_{2} \times \frac{{m}_{s}}{{m}_{r}} + {β}_{3} \times \frac{{q}_{s}}{{q}_{r}} + {β}_{4} \times \frac{{I}_{s}}{{I}_{r}} & [ Equation 4 ]\end{matrix}$$

Eₚ in Equation 3 may be calculated by Equation 4. In Equation 4, β₁, β₂, β₃, and β₄ are values measured to be different for each user, b is a weight, m is muscle strength, q is body fat, and 1 is a level of physical activity. p is a personal number, s is a current state, and r is a recommended state. In Equation 4, q may be calculated by Equation 5, and 1 may be calculated by Equation 6.

a=α₁×a₁+α₂×a₂ [Equation 5]

l=y₁×l₁+y₂×l₂+y₃×l₃ [Equation 6]

In Equation 5, α₁ and α₂ are values measured to be different for each user, q₁ is the weight of body fat, and q₂ denotes the weight of muscle. In Equation 6, y₁, y₂, and y₃ are values measured to be different for each user, l₁ is the amount of work done, l₂ is a lung capacity, and l₃ is a respiratory rate.

As described above, the exercise management unit 380 may assess the exercise score and the degree of exercise to thereby track and manage the amount of exercise of the user and provide the user with the exercise score and the degree of exercise. In addition, the exercise management unit 380 may provide the external institution 500 with the exercise score and the degree of exercise such that the user receives a customized exercise service and a physical strength management service from the external institution 500.

The expression for calculating the exercise score may be referred to as a personal exercise activity index (PEAI) but is not limited to Equation 3.

FIG. 7 is an operational flowchart showing a method of assessing the degree of absolute stress according to the present invention.

The device 300 for health information prediction may include the absolute stress management unit 385 configured to determine the degree of absolute stress on the basis of stress information of the user. The method of assessing absolute stress shown in FIG. 7 may be performed by the absolute stress management unit 385 shown in FIG. 2, but the agent of the operation is not limited thereto.

The method of assessing the degree of stress using the absolute stress management unit 385 includes collecting stress information of the user from the open-type PHR platform 100 or the directly servicing PHR platform (S710) and calculating an absolute stress score according to a designated expression shown in Equation 7 (S720), determining whether the absolute stress score is less than or equal to 100 (S730), estimating the stress to be stable when the absolute stress score is less than or equal to 100 (S740), and estimating the stress to be excessive when the absolute stress score is greater than 100 (S750).

$$\begin{matrix}absolute\_stress \_score = {AS}_{p} \times 100 & [ Equation 7 ] \\ {AS}_{p} = {β}_{p} \times \frac{{hr}_{1} + {hr}_{2} + … + {hr}_{n}}{n \times {hr}_{s}} & [ Equation 8 ]\end{matrix}$$

In Equation 7, ASₚ may be calculated through Equation 8. In Equation 8, βₚ is a value measured to be different for each user, hrₙ is a heart rate in nth data, hrₛ is a heart rate in a stable situation, and p is a personal number.

As described above, the absolute stress management unit 385 may assess the absolute stress score and the degree of stress to thereby perform a working environment management and a mental health management of the user for each job and each division and provide the user with the absolute stress score and the degree of stress. In addition, the absolute stress management unit 385 may provide the external institution 500 with the absolute stress score and the degree of stress such that the user receives a mental care service from the external institution 500.

The expression for calculating the absolute stress score may be referred to as a personal absolute stress index (PAST) but is not limited to Equation 7.

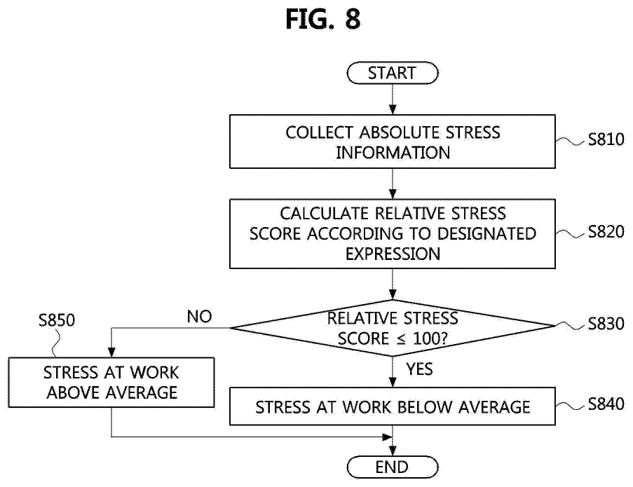

FIG. 8 is an operational flowchart showing a method of assessing the degree of relative stress according to the present invention.

The device 300 for health information prediction may include the relative stress management unit 390 configured to determine the degree of relative stress on the basis of the absolute stress score. The method of assessing the degree of relative stress shown in FIG. 8 may be performed by the relative stress management unit 390 shown in FIG. 2. However, the agent of the operation is not limited thereto.

The method of assessing the degree of stress using the relative stress management unit 390 includes collecting the absolute stress score of the user from the absolute stress management unit 385 (S810), calculating a relative stress score according to a designated expression as in Equation 9 (S820), determining whether the relative stress score is less than or equal to 100 (S830), estimating the stress to be below the average when the relative stress score is less than or equal to 100 (S840), and estimating the stress to be above the average when the relative stress score is greater than 100 (S850).

$$\begin{matrix}relative\_stress \_score = {RS}_{p} \times 100 & [ Equation 9 ] \\ {RS}_{p} = \frac{n \times {AS}_{p}}{{AS}_{1} + {AS}_{2} + … {AS}_{p} + … + {AS}_{n}} & [ Equation 10 ]\end{matrix}$$

In Equation 9, RSₚ may be calculated through Equation 10. In Equation 10, AS is an absolute stress score, p is a personal number, and n is a total number of people in a company.

As described above, the relative stress management unit 390 may assess the relative stress score and the degree of stress to thereby perform a working environment management and a mental health management of the user for each job and each division and provide the user with the relative stress score and the degree of stress. In addition, the relative stress management unit 390 may provide the external institution 500 with the relative stress score and the degree of stress such that the user receives a mental care service and the like from the external institution 500.

The expression for calculating the relative stress score may be referred to as a personal relative stress index (PRSI) but is not limited to Equation 9.

The operations of the methods according to the embodiments may be embodied as computer-readable programs or codes on a computer-readable recording medium. The computer-readable recording medium is any data storage device that can store data that can be thereafter read by a computer system. In addition, the computer-readable recording medium may also be distributed over network-linked computer systems in which the computer-readable program or code is stored and executed in a distributed fashion.

In addition, the computer-readable recording medium may include a hardware device specially constructed to store and execute a program command, for example, a read-only memory (ROM), a random-access memory (RAM), and a flash memory. The program command may include a high-level language code executable by a computer through an interpreter in addition to a machine language code made by a compiler

Some aspects of the present invention have been described in the context of the apparatus but may represent description of a method corresponding thereto, and a block or an apparatus corresponds to an operation of a method or a feature thereof. Similarly, some aspects having been described in the context of the method may also be represented by a block or items corresponding to the method or a feature of an apparatus corresponding to the method. Some or all of the operations of the method may be performed, for example, by (or using) the hardware device, such as a microprocessor, a programmable computer, or an electronic circuit. In some embodiments, one or more of most important operations of the method may be performed by such a device.

In some embodiments, a programmable logic device (for example, a field programmable gate array) may be used to perform some or all of the above described functions of the methods. In some embodiments, a field programmable gate array may operate together with a microprocessor to perform one of the above described methods. In general, the methods may be performed by any hardware device.

1. A device for predicting health information using an open-type personal health record (PHR) platform, the device comprising:
a health examination unit configured to derive health examination data from the open-type PHR platform;
a diagnosis unit configured to derive diagnostic data from the open-type PHR platform; and
a health score calculation unit configured to assess a disease-specific score determinant from the open-type PHR platform, calculate a disease-specific health score according to the disease-specific score determinant on the basis of the health examination data and the diagnostic data, and calculate a personal health score according to the disease-specific health score.

2. The device of claim 1, further comprising a future health score calculation unit configured to calculate a disease-specific future health score according to the disease-specific score determinant on the basis of the health examination data and the diagnostic data.

3. The device of claim 2, further comprising a health management unit configured to derive an individual health indicator on the basis of the personal health score and the disease-specific future health score.

4. The device of claim 1, further comprising a comparison analysis unit configured to perform comparison and analysis on an environment group similar to a user on the basis of the disease-specific health score.

5. The device of claim 1, further comprising a service evaluation unit configured to derive medical service evaluation information of a medical institution from the open-type PHR platform.

6. The device of claim 1, further comprising a personal health record (PHR) platform including:
an information collecting unit configured to collect health and disease management knowledge;
a feedback unit configured to perform feedback with reference to a knowledge storage;
a personal life health information record interface;
an application programming interface (API) for service system integration and linkage;
a data linking unit configured to link typical data and atypical data;
a cloud service unit configured to provide a cloud computing service using big data; and
a health predicting unit configured to predict individual health care through converting a personal health record into public data.

7. The device of claim 1, further comprising:
a u-health care unit configured to provide real time u-health care using health information related data;
a protected health information (PHI) monitor unit configured to provide monitoring of PHI using health insurance big data;
a disease prevention unit configured to provide a disease prevention program through analysis of disease data;
a medical service unit configured to provide a health and medical service including a diagnosis, a treatment, and follow-up care using the u-health care;
a data management unit configured to perform integrated data management for providing a recommended service through a vaccination time and existing personal health record material that is linked with vaccination data;
a prediction model unit configured to provide a health-prediction-likelihood model through analysis of a health examination result, a treatment, and an administration history; and
a disease analysis unit configured to analyze a probability of a disease outbreak through integration of personal genetic information and health type information.

8. The device of claim 1, further comprising an exercise management unit configured to collect physical information and exercise information of a user, calculate an exercise score on the basis of the physical information and the exercise information, and assess a degree of exercise of the user according to the exercise score.

9. The device of claim 1, further comprising an absolute stress management unit configured to collect stress information of a user, calculate an absolute stress score on the basis of the stress information, and assess a degree of stress of the user according to the absolute stress score.

10. The device of claim 9, further comprising a relative stress management unit configured to calculate a relative stress score on the basis of the absolute stress score and assess a degree of stress of the user according to the relative stress score.

11. A method of predicting health information using an open-type personal health record (PHR) platform, the method comprising:
deriving health examination data, diagnostic data, and a disease-specific score determinant from the open-type PHR platform; and
calculating a disease-specific health score according to the disease-specific score determinant on the basis of the health examination data and the diagnostic data.

12. The method of claim 11, further comprising calculating a personal health score according to the disease-specific health score.

13. The method of claim 11, further comprising calculating a disease-specific future health score according to the disease-specific score determinant on the basis of the health examination data and the diagnostic data.

14. The method of claim 13, further comprising deriving an individual health indicator on the basis of the disease-specific health score and the disease-specific future health score.

15. The method of claim 11, further comprising performing comparison and analysis on an environment group similar to a user on the basis of the disease-specific health score.

16. The method of claim 11, further comprising deriving medical service evaluation data of a medical institution from the open-type PHR platform.

17. The method of claim 11, further comprising constructing a personal health record (PHR) platform, wherein the constructing of the PHR platform includes:
collecting health and disease knowledge;
performing feedback with reference to a knowledge storage;
using a personal life health information record interface;
using an application programming interface (API) for service system integration and linkage;
linking typical data and atypical data;
providing a cloud computing service using big data; and
predicting individual health care through converting a personal health record into public data.

18. The method of claim 11, further comprising:
providing real time u-health care using health information related data;
monitoring protected health information (PHI) using health insurance big data;
providing a disease prevention program through analysis of disease data;
providing a health and medical service including a diagnosis, a treatment, and follow-up care using the u-health care;
providing integrated data management for providing a recommended service through a vaccination time and existing personal health record material that is linked with vaccination data;
providing a health-prediction-likelihood model through analysis of a health examination result, a treatment, and an administration history; and
analyzing a probability of a disease outbreak through integration of personal genetic information and health type information.

19. The method of claim 11, further comprising:
collecting physical information and exercise information of a user;
calculating an exercise score on the basis of the physical information and the exercise information; and
assessing a degree of exercise of the user according to the exercise score.

20. The method of claim 11, further comprising:
collecting stress information of a user;
calculating an absolute stress score on the basis of the stress information; and
assessing a degree of stress of the user according to the absolute stress score.

21. The method of claim 20, further comprising:
calculating a relative stress score on the basis of the absolute stress score; and
assessing a degree of stress of the user according to the relative stress score.
